(19) United States
(12) Patent Application Publication
(10) Pub. No.: US 2026/0192111 A1
(43) Pub. Date: Jul. 9, 2026

(54) METHOD AND APPARATUS FOR ELECTRICAL STIMULATION OF THE VAGUS NERVE

(71) Applicant: Tivic Health Systems Inc., San Antonio, TX (US)

(72) Inventors: Blake Taylor Gurfein, San Rafael, CA (US); Carolyn Roberta McMakin, Vancouver, WA (US)

(21) Appl. No.: 19/305,936

(22) Filed: Aug. 21, 2025

Related U.S. Application Data
(63) Continuation of application No. PCT/US2024/021789, filed on Mar. 27, 2024.
(60) Provisional application No. 63/492,402, filed on Mar. 27, 2023.

(51) Int. Cl. A61N 1/36; A61N 1/02; A61N 1/04

(52) U.S. Cl. CPC A61N 1/36031; A61N 1/025; A61N 1/0456; A61N 1/0476; A61N 1/0492; A61N 1/36034

(57) ABSTRACT

Methods for activating a person's autonomic nervous system may include: electrically coupling a first electrode to a vagus nerve via a transdermal coupling at a location rostral to the human's thoracic spine; electrically coupling a second electrode to a nerve in communication with the same vagus nerve or a different vagus nerve via a transdermal coupling at a site caudal to the human's cervical spine; and applying a therapeutic current waveform to cause current flow between the first and second electrodes and along at least a portion of the vagus nerve or nerves between the first and second electrodes. Apparatuses are disclosed for performing the methods.

### CROSS REFERENCE TO RELATED APPLICATIONS

The present application is a Continuation Application which claims priority benefit under 35 U.S.C. § 120 from the co-pending International Patent Application No. PCT/US2024/021789, entitled “Method and Apparatus for Electrical Stimulation of the Vagus Nerve,” filed Mar. 27, 2024 (Docket No. 3048-048-04). International Patent Application No. PCT/US2024/021789 claims priority benefit from U.S. Provisional Patent Application No. 63/492,402, entitled “Method and Apparatus for Electrical Stimulation of the Vagus Nerve,” filed Mar. 27, 2023 (Docket Number 3048-048-02). The foregoing applications, to the extent not inconsistent with the disclosure herein, are incorporated by reference.

### SUMMARY

According to an embodiment, an apparatus for applying a therapeutic current waveform to a human vagus nerve includes a waveform generator configured to produce: at a first terminal, a first therapeutic current waveform selected for application to a human subject via a first electrode transdermally coupled at a first location rostral to the subject's thoracic spine, at a second terminal, a second therapeutic current waveform selected for application to the human subject via a second electrode transdermally coupled at a second location rostral to the subject's thoracic spine, at a third terminal, a ground potential configured to be electrically coupled to one or more electrodes transdermally coupled at respective locations caudal to the subject's cervical spine; and a logic circuit configured to control operation of the waveform generator.

According to an embodiment, an apparatus for applying a therapeutic current waveform to a human vagus nerve includes a housing, a logic circuit disposed in the housing, a waveform generator operatively coupled to the logic circuit and disposed in the housing, and one or more electrode connectors disposed in a wall of the housing and operatively coupled to the waveform generator, the one or more electrode interfaces being configured to couple to adhesive electrodes, the adhesive electrodes being configured for coupling to respective locations on human skin for transdermally applying the therapeutic current waveform along at least a first vagus nerve of the human. The waveform generator is configured to generate a therapeutic current waveform responsive to the logic circuit. The logic circuit may be configured to control the waveform generator to generate a sequence of different frequency therapeutic current waveforms selected to cause a parasympathetic response in an autonomic nervous system of the human.

According to an embodiment, an apparatus for applying a therapeutic current waveform to a human vagus nerve includes a housing, a logic circuit disposed in the housing, and a waveform generator operatively coupled to the logic circuit and disposed in the housing, the waveform generator being configured to generate a therapeutic current waveform responsive to the logic circuit. One or more electrode connectors are disposed in a wall of the housing and operatively coupled to the waveform generator, the one or more electrode connectors being configured to couple to adhesive electrodes configured for coupling to respective locations on human skin for transdermally applying the therapeutic current waveform along first and second vagus nerves of the human. The logic circuit is configured to control the waveform generator to generate a first therapeutic current waveform coupled at least through the adhesive electrode disposed on a first lateral side of the human rostral to the human's thoracic spine and to generate a second therapeutic current waveform, different from the first therapeutic current waveform, coupled at least through the adhesive electrode disposed on a second lateral side of the human opposite to the first lateral side of the human rostral to the human's thoracic spine, to one or more adhesive electrodes disposed caudal to the human's cervical spine. The first and second waveforms may be characterized by different frequencies that are applied simultaneously.

According to an embodiment, a method for operating a vagus nerve therapeutic current stimulation apparatus configured to activate an autonomic nervous system response in a human includes applying a therapeutic current waveform between a first electrode transdermally coupled at a location rostral to a human's thoracic spine and a second electrode transdermally coupled at a location caudal to the human's cervical spine, controlling the therapeutic current waveform to have a peak current of between 100 and 300 microamps, receiving a signal from a sensor, and regulating one or more characteristics of the therapeutic current waveform in response to the signal from the sensor.

According to an embodiment, a method for operating a vagus nerve electrical current stimulation apparatus configured to activate an autonomic nervous system response in a human includes electrically coupling a first electrode to a human subject at a location rostral to the subject's thoracic spine, electrically coupling a second electrode to the human subject at a location caudal to the subject's cervical spine, applying a therapeutic current waveform between the first electrode and the second electrode, controlling the therapeutic current waveform to have a peak current of between 100 and 300 microamps, receiving a signal from a sensor, and regulating one or more characteristics of the therapeutic current waveform in response to the signal from the sensor.

According to an embodiment, a method for operating a vagus nerve therapeutic current stimulation apparatus configured to activate an autonomic nervous system response in a human includes measuring an impedance between first and second electrodes with a therapeutic current generation circuit, the impedance being consistent with electrically coupling the first electrode to a first vagus nerve via a transdermal coupling at a location rostral to the human's thoracic spine and electrically coupling the second electrode to a nerve in electrical communication with the first vagus nerve or a second vagus nerve, via a transdermal coupling at a site caudal to the human's cervical spine, receiving a control input into a control circuit operatively coupled to the therapeutic current generation circuit, the control input being indicative that a therapeutic current waveform is to be applied between the first and second electrodes, and generating the therapeutic current waveform to cause current flow between the first and second electrodes along and through at least a portion of the first vagus nerve or first and second vagus nerves between the first and second electrodes.

According to an embodiment, a method for activating an autonomic nervous system response in a human includes electrically coupling a first electrode to a vagus nerve via a transdermal coupling at a location rostral to the human's thoracic spine. The method includes electrically coupling a second electrode to a nerve in electrical communication with the same vagus nerve or a different vagus nerve of two, via a transdermal coupling at a site caudal to the human's cervical spine. A therapeutic waveform is applied to cause current flow between the first and second electrodes along and through at least a portion of the vagus nerve or nerves between the first and second electrodes.

According to an embodiment, a method for controlling a therapeutic waveform applied along human vagus nerves associated with the autonomic nervous system of a human includes arranging first and second electrodes to operatively couple along at least a portion of the vagus nerve(s) between the first and second electrodes. A therapeutic waveform is applied with a waveform generator to cause an alternating polarity current flow between the first and second electrodes. The method includes measuring a cardiac function of the human with a cardiac function sensor. A control signal or control data is output to a waveform generator to cause modification of the therapeutic waveform if the measured cardiac function is a predetermined amount different from a baseline cardiac function.

### BRIEF DESCRIPTION OF THE DRAWINGS

FIG. 1 is a flowchart showing a method for activating an autonomic nervous system response in a human, according to an embodiment.

FIG. 2 is a diagram of an apparatus for activating an autonomic nervous system response, according to an embodiment.

FIG. 3 is a diagram of an apparatus for activating an autonomic nervous system response, according to another embodiment.

FIG. 4 is a diagram of an apparatus for activating an autonomic nervous system response, according to another embodiment.

FIG. 5 is a diagram of an apparatus for activating an autonomic nervous system response, according to another embodiment.

FIG. 6 is a diagram of an apparatus for activating an autonomic nervous system response, according to another embodiment.

FIG. 7 is a diagram of a system for activating an autonomic nervous system response, according to another embodiment.

FIG. 8 is a diagram of a system for activating an autonomic nervous system response, according to another embodiment.

FIG. 9 is a chart showing the left and right mean pupil size of test subjects over the course of a test battery employing devices and methods described with reference to disclosed embodiments.

FIG. 10 is a chart showing measured responses of heartrate variability (HRV) to the test battery for all subjects.

FIG. 11 is a chart showing measured responses of HRV to the test battery for responsive group subjects.

FIG. 12 is a chart showing normalized responses of HRV to the test battery for all subjects.

FIG. 13 is a chart showing normalized responses of HRV to the test battery for responsive group subjects.

FIG. 14 is a set of charts showing normalized changes in EEG passband power measured for frontal right (FLR) and frontal left (FLL) lobe positions.

FIG. 15 is a set of charts showing normalized changes in EEG passband power measured for parietal (P), temporal (TEMP), and occipital (OC) lobe positions, with separate graphs for left (L) and right (R) respective lobes.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description and drawings are not meant to be limiting. Other embodiments may be utilized, and other changes may be made, without departing from the spirit or scope of the subject matter presented here, which shall be limited only by the claims.

The human vagus nerve is the longest nerve in the human body. It is bilateral, meaning there are separate branches on the right and left side of a human body. The vagus nerve is a nerve bundle that is about 25% efferent (transmission of signals from the brain to the body) and 75% afferent (transmission of signals from the body to the brain). Studies by the applicant have indicated that application of therapeutic current signals, described herein, along the vagus nerve(s) affect the sympathetic/parasympathetic state of the autonomic nervous system. It is believed that such treatment may, in turn, affect mood, vitality, and organ function, reduce inflammation, and possibly slow disease progression. Moreover, recent studies show application of therapeutic current waveforms to affect brain activity in positive ways akin to meditation and psych drugs.

For economy of language, description and claims herein will generally refer to “the vagus nerve”. As is known to those having knowledge in the art, the human body includes two vagus nerves on opposite sides of the body that are in electrical communication with one another. For arrangements where electrodes are operatively coupled to laterally opposite sides of the body, it will be understood that a given electrode will be most closely operatively coupled to the vagus nerve nearest to the electrode, and therapeutic waveforms applied between the electrode pair may travel primarily along one or the other of the vagus nerves, or may travel along both vagus nerves, with either current path being completed through cross-connections between the two vagus nerves. The inventors understand that when a nerve is disposed parallel to a current flow path through the body, the current travels primarily along and through nerves (rather than surrounding tissues) owing to the relatively low electrical resistance of nerves compared to the electrical resistance of other bodily tissues.

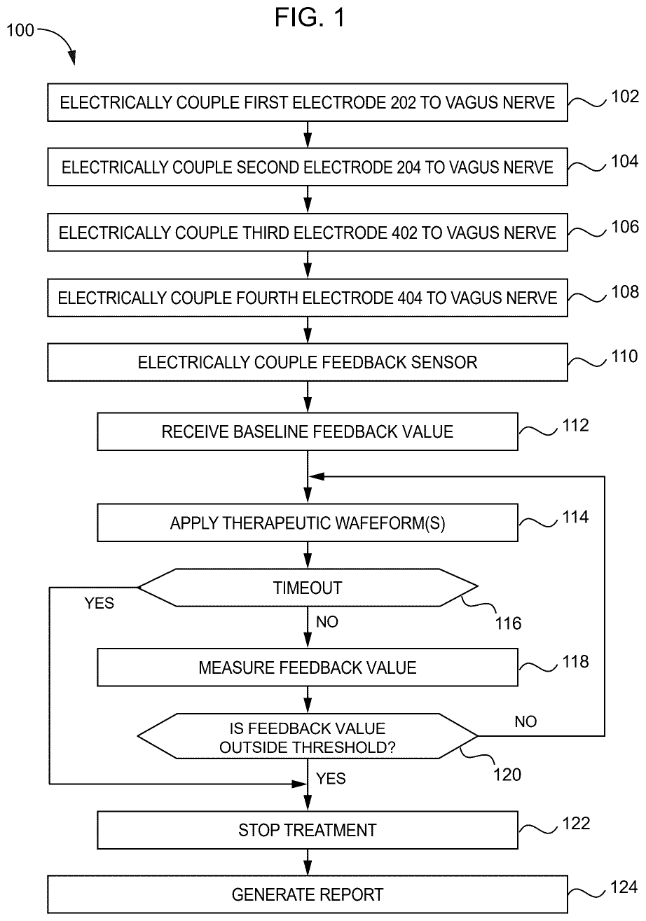

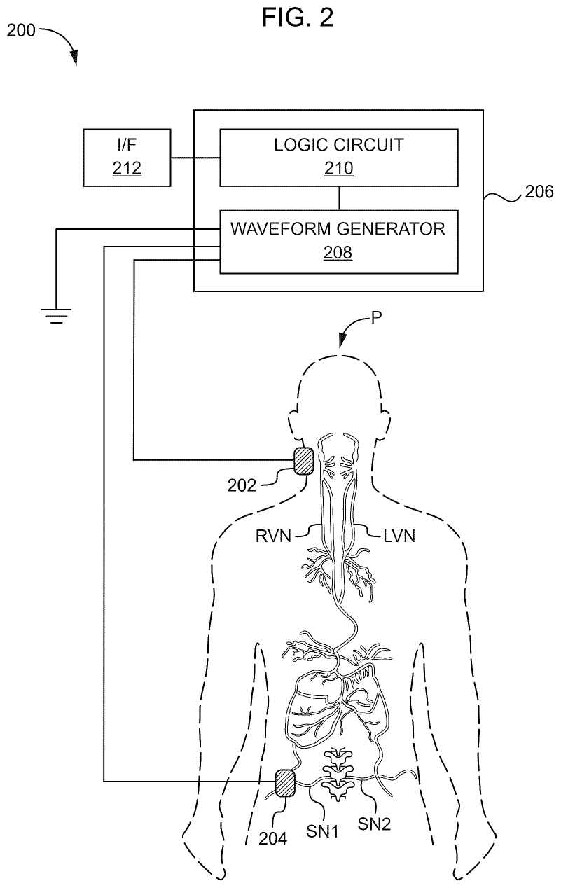

FIG. 1 is a flowchart showing a method 100 for activating an autonomic nervous system response in a human, according to an embodiment. FIG. 2 is a diagram 200 of an apparatus for activating an autonomic nervous system response, according to an embodiment. Referring to FIGS. 2 through 6, the method 100 for activating an autonomic nervous system response in a human includes, in step 102, electrically coupling a first electrode to a vagus nerve via a transdermal coupling at a location rostral to the human's thoracic spine (e.g., see FIG. 2, 202). Step 104 includes electrically coupling a second electrode to a nerve in electrical communication with the same vagus nerve or a different vagus nerve of two, via a transdermal coupling at a site caudal to the human's cervical spine; (e.g., see FIG. 2, 204). Step 114 includes applying a therapeutic waveform to cause current flow between the first and second electrodes 202, 204 along and through at least a portion of the vagus nerve or nerves between the first and second electrodes.

Electrically coupling the first electrode 202 to the vagus nerve, in step 102, may include electrically coupling the first electrode 202 to the vagus nerve via a transdermal coupling to a human cervical nerve in electrical communication with the vagus nerve. Electrically coupling the first electrode 202 to the vagus nerve, in step 102, may include electrically coupling the first electrode 202 to the vagus nerve via a transdermal coupling to a human cranial nerve. Electrically coupling the first electrode 202 to the vagus nerve, in step 102, may include electrically coupling the first electrode 202 to the vagus nerve via a transdermal coupling superjacent to an auricular nerve. Electrically coupling the first electrode 202 to the vagus nerve, in step 102, may include electrically coupling the first electrode 202 to the vagus nerve via a transdermal coupling to a submandibular ganglion (e.g., N VII).

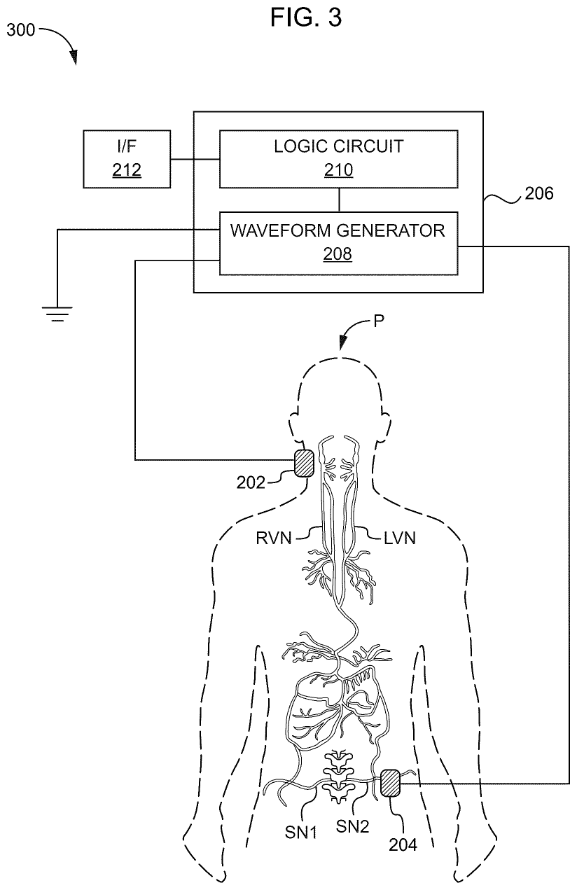

According to an embodiment, shown in FIG. 2, electrically coupling the second electrode 204 to the nerve in electrical communication with the vagus nerve, in step 104, may include electrically coupling the second electrode 204 to the same vagus nerve by applying an adhesive electrode to the same lateral side of the human. In another embodiment, shown in FIG. 3, electrically coupling the second electrode 204 to the nerve in electrical communication with the vagus nerve, in step 104, may include electrically coupling the second electrode 204 to a different vagus nerve than the first electrode 202 by applying an adhesive electrode to the opposite lateral side of the human.

Electrically coupling the second electrode 204 to the nerve in electrical communication with the vagus nerve, in step 104, may include electrically coupling the second electrode 204 to the human vagus nerve via a transdermal coupling at a location caudal the human rib cage. Electrically coupling the second electrode 204 to the nerve in electrical communication with the vagus nerve may include electrically coupling the second electrode 204 to an autonomic plexus associated with an organ of the human.

FIG. 3 is diagram 300 of an apparatus for activating an autonomic nervous system response, according to another embodiment. Referring to FIGS. 1 and 3, according to an embodiment, electrically coupling the first electrode 202 to the vagus nerve via the transdermal coupling at the location rostral to the human's thoracic spine, in step 102, includes electrically coupling the first electrode to one of a laterally right side or a laterally left side of the human. Electrically coupling the second electrode 204 to the nerve in electrical communication with the vagus nerve via the transdermal coupling at the site caudal to the human's cervical spine, in step 104, includes coupling the second electrode to one of the left side or the right side of the human opposite to the side to which the first electrode is coupled. Electrically coupling the second electrode 204 includes electrically coupling the second to the different vagus nerve of two relative to the first electrode 202. Electrically coupling a second electrode 204 to the nerve in electrical communication with the vagus nerve via the transdermal coupling at the site caudal to the human's cervical spine includes coupling the second electrode 204 to an autonomic plexus associated with an organ of the human and in electrical communication with the vagus nerve.

Applying the therapeutic waveform, in step 114, may include applying a first therapeutic waveform including an alternating current waveform having a frequency. Applying the first therapeutic waveform, in step 114, may include applying an alternating current waveform having a frequency between 8 hertz and 1000 hertz. Applying the first therapeutic waveform may include, in step 114, applying the alternating current waveform having a frequency between 9 hertz and 30 hertz.

Applying the first therapeutic waveform, in step 114, may include applying the first therapeutic waveform having at least one frequency selected from the group consisting of 9 hertz, 22 hertz, 35 hertz, 40 hertz, 45 hertz, 49 hertz, 57 hertz, 62 hertz, 71 hertz, 77 hertz, 81 hertz, 124 hertz, 142 hertz, 294 hertz, 321 hertz, 900 hertz, 920 hertz, and 970 hertz, each frequency having a tolerance of plus or minus 2 hertz. Additionally or alternatively, applying the first therapeutic waveform, in step 114, may include applying the first therapeutic waveform having at least one frequency selected from the group consisting of 9 hertz, 40 hertz, 49 hertz, 57 hertz, 81 hertz, 124 hertz, 294 hertz, 321 hertz, 900 hertz, 920 hertz, and 970 hertz. Applying the first therapeutic waveform, in step 114, may include applying the first therapeutic waveform having at least one frequency selected from the group consisting of 22 hertz, 35 hertz, 45 hertz, 62 hertz, 71 hertz, 77 hertz, and 142 hertz. Applying the first therapeutic waveform, in step 114, may include applying the first therapeutic waveform having at least one frequency selected from the group consisting of 9 hertz, 22 hertz, 35 hertz, 40 hertz, 45 hertz, 49 hertz, 57 hertz, 62 hertz, 71 hertz, 77 hertz, 81 hertz, 124 hertz, 142 hertz, 294 hertz, 321 hertz, 900 hertz, 920 hertz, and 970 hertz, each frequency having a tolerance of plus or minus 1 hertz.

According to an embodiment, applying the first therapeutic waveform, in step 114, includes applying a sinusoidal alternating current waveform. Alternatively, applying the first therapeutic waveform, in step 114 may include applying a waveform including alternating polarity voltage spikes. According to an embodiment, applying the first therapeutic waveform, in step 114 may include applying a waveform including alternating polarity voltage spikes having a duty cycle below 5%. Applying the first therapeutic waveform, in step 114, may include applying a waveform comprising alternating polarity voltage spikes having a duty cycle of about 2%. Applying the first therapeutic waveform, in step 114, may include applying a waveform having a constant peak current. Applying the first therapeutic waveform, in step 114, may include monitoring current and responsively modifying driving of the first therapeutic waveform to maintain the constant peak current.

According to an embodiment, applying the therapeutic waveform, in step 114, includes driving the first therapeutic waveform with a waveform generator (see 208). The method 100 includes detecting an amount of current delivered. The method 100 includes amplifying or attenuating the waveform generator 208 to output constant electrical current.

Applying the therapeutic waveform, in step 114, may include driving the first therapeutic waveform with a waveform generator 208. Adjusting the waveform generator 208 to output a reduced voltage as a function of a number of cycles, such that applying the first therapeutic waveform, in step 114, may include maintaining constant peak current as electrical impedance through the human body decreases, the decrease being associated with the number of previous cycles.

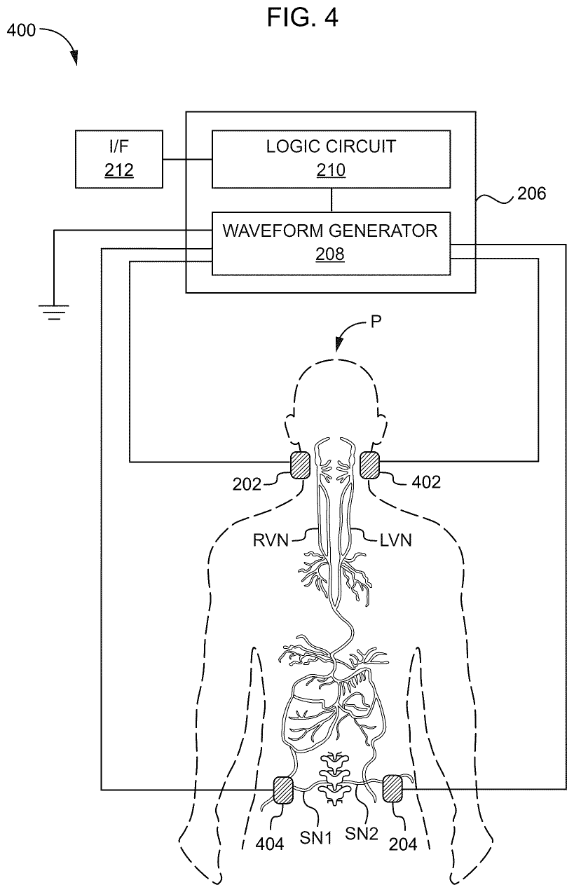

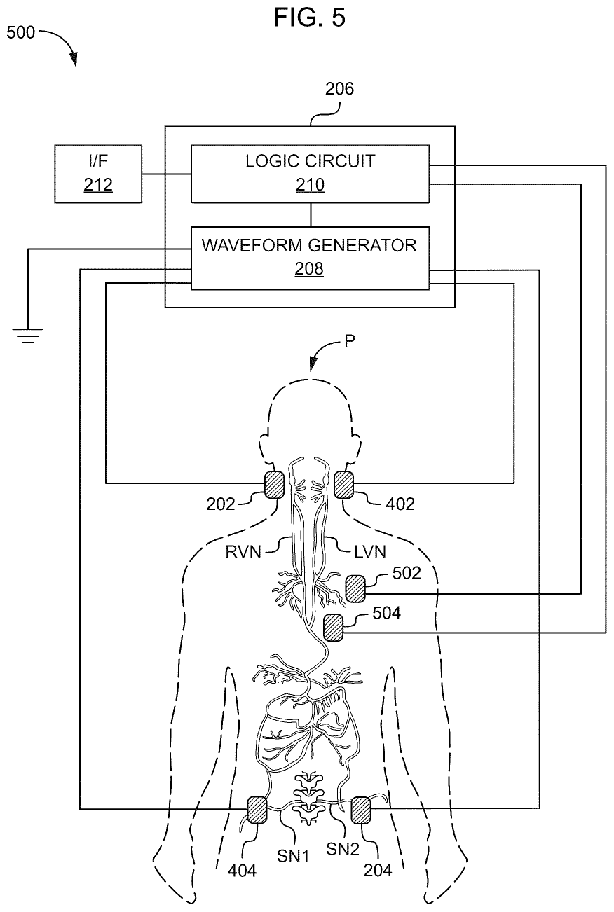

The method 100 may further include receiving a user input via an interface (I/F) 212 into a logic circuit 210, the user input may correspond to at least one of a condition to be treated and a therapeutic waveform schedule. Receiving the therapeutic waveform schedule into the logic circuit 210 may include receiving a waveform frequency. Receiving the therapeutic waveform schedule into the logic circuit 210 may include receiving two waveform frequencies. The method 100 may include loading corresponding parameters into the waveform generator 208 to cause the waveform generator 208 to cooperate with four electrodes 202, 204, 402, 404 electrically coupled to the vagus nerve(s) as shown in FIG. 4. The four electrodes may be disposed as cross-body pairs including the first pair 202, 204 and a second pair 402, 404, to apply a first push-pull waveform frequency (f₁) to the first pair of electrodes 202, 204 and, via the electrical couplings, through portions of two respective vagus nerves, and apply a second push-pull waveform frequency (f₂) to the second pair of electrodes. Each of two frequencies f₁ and f₂ may be determined responsive to the condition to be treated or the therapeutic waveform schedule received into the logic circuit 210 from the interface (I/F) 212 (steps not shown in FIG. 1, see FIG. 5). The waveform generator 208 is configured to communicate with the first pair of electrodes 202, 204 and with the second pair of electrodes 402, 404 to apply a first waveform having a first frequency to the vagus nerve(s) via the first pair of electrodes 202, 204 and apply a second waveform having a second frequency to the vagus nerve(s) via the second pair of electrodes 402, 404. FIG. 4 is a diagram 400 of an apparatus for activating an autonomic nervous system response, according to another embodiment. FIG. 5 is a diagram 500 of an apparatus for activating an autonomic nervous system response, according to another embodiment.

Applying the first therapeutic waveform, in step 114, may include applying an alternating current waveform with one of the first and second electrodes 202, 204 and holding the other of the first and second electrodes 202, 204 at ground. Applying the first therapeutic waveform, in step 114, may include applying an alternating current waveform having a first polarity with the first electrode 202 and applying a synchronized alternating waveform at a second polarity opposite to the first polarity with the second electrode 204 (such as in a push-pull arrangement).

The different vagus nerve may include a second vagus nerve, and may include electrically coupling a third electrode 402 to the second vagus nerve of the human via a transdermal coupling at a third location on an opposite side of the human relative to the first electrode 202 and location. Electrically coupling a fourth electrode 404 to a nerve in electrical communication with the vagus nerve, via a transdermal coupling, at a fourth site on an opposite side relative to the second electrode. Applying a second therapeutic waveform may cause current flow between the third and fourth electrodes 402, 404 and along at least a portion of the vagus nerve.

Applying the second therapeutic waveform may include applying a waveform having a second frequency, different from a first frequency applied between the first and second electrodes 202, 204, to cause current flow between the third and fourth electrodes 402, 404. One of the third and fourth electrodes 402, 404 applies the second therapeutic waveform while the other of the third and fourth electrodes 402, 404 is at a signal ground. The second electrode 204 may apply the therapeutic waveform at a polarity opposite to the polarity of the therapeutic waveform applied by the first electrode 202. The fourth electrode 404 may apply the second therapeutic waveform at a polarity opposite to the polarity of the second therapeutic waveform applied by the third electrode 402.

The frequency of the therapeutic waveform applied between the first and second electrodes 202, 204 may be a different frequency relative to the frequency of the second therapeutic waveform applied between the third and fourth electrodes 402, 404. The frequency of the therapeutic waveform applied between the first and second electrodes 202, 204 may be selected from the group consisting of 9 hertz, 40 hertz, 49 hertz, 57 hertz, 81 hertz, 124 hertz, 294 hertz, 321 hertz, 900 hertz, 920 hertz, and 970 hertz. The frequency of the second therapeutic waveform applied between the third and fourth electrodes 402, 404 may be selected from the group consisting of 22 hertz, 35 hertz, 45 hertz, 62 hertz, 71 hertz, 77 hertz, and 142 hertz.

The method 100 may include measuring a cardiac function of the human with a cardiac function sensor, for example, disposed in the logic circuit, see 210. The method 100 may include outputting a control signal or control data to a therapeutic waveform generator 208 to cause modification of the therapeutic waveform if the measured cardiac function is a predetermined amount different from a baseline cardiac function.

According to an embodiment, a method for controlling a therapeutic waveform applied along human vagus nerves associated with the autonomic nervous system of a human includes arranging first and second electrodes 202, 204 to operatively couple along at least a portion of the vagus nerve(s) between the first and second electrodes 202, 204. The method includes applying a therapeutic waveform with a waveform generator 208 to cause an alternating polarity current flow between the first and second electrodes 202, 204. The method includes measuring a cardiac function of the human with a cardiac function sensor. The method 100 includes outputting a control signal or control data to a waveform generator 208 to cause modification of the therapeutic waveform if the measured cardiac function is a predetermined amount different from a baseline cardiac function.

The method 100 may further include outputting the measured cardiac function as an image on an electronic display for reading by a therapist. Control input may be received from the therapist via a human interface operatively coupled to a therapeutic waveform generator 208, the control input may be arranged to correspond to the control signal or control data.

The method may include operation with an electronic controller 206 operatively coupled to the waveform generator 208 and the cardiac function sensor. The measured cardiac function may be compared to a baseline cardiac function of the human. The control signal or control data may be output to the waveform generator 208.

Measuring the cardiac function in the human may include measuring heart rate. Measuring the cardiac function in the human may include measuring blood pressure. Measuring the cardiac function in the human may include measuring blood oxygenation. Measuring the cardiac function may include measuring a heart rate variability (HRV) in the human. Comparing the measured cardiac function to the baseline cardiac function may include comparing the measured HRV to a baseline heart rate variability of the human. Outputting the control signal or control data to cause modification of the therapeutic waveform if the measured cardiac function is a predetermined amount different than the baseline cardiac function may include outputting the control signal or control data to cause modification of the therapeutic waveform if the measured HRV is a predetermined amount different than the baseline HRV. The HRV may be a root mean square of successive differences in heart rate. The HRV may be a root mean square of successive differences in intervals between successive heartbeats.

The method 100 may include comparing the measured cardiac function to the baseline cardiac function with an electronic controller 206. The one or more control signals or data may be determined with the electronic controller 206. Modifying the therapeutic waveform as a function of the measured cardiac function may include outputting the one or more control signals or data from the electronic controller 206 to the waveform generator 208. Modifying the therapeutic waveform may include changing an amplitude of the therapeutic waveform. Modifying the therapeutic waveform may include changing a frequency of the therapeutic waveform. Modifying the therapeutic waveform may include stopping application of the therapeutic waveform. Modifying the therapeutic waveform may include prompting a therapist to move the second electrode 204 of the first and second electrodes 202, 204 to a different position to electrically couple to the human vagus nerve via a transdermal coupling to an autonomic plexus associated with an organ of the human at the different location below the human rib cage. At least one of the first and the second electrodes 202, 204 may include an addressable array of electrical coupling points. Modifying the therapeutic waveform may include causing the current to flow through a different point in the addressable array of electrical coupling points.

With reference to FIG. 2, according to an embodiment, a laterally right-side vagus nerve RVN passes from the cranium down the neck of a human person P into the body cavity, where the vagus nerve RVN may be electrically or neuronally connected to communicate with its complementary left-side vagus nerve LVN, as well as being electrically and neuronally connected to communicate with a spinal nerve SN1 or SN2. The spinal nerves SN may be electrically or neuronally connected to at least one of the two vagus nerves RVN and LVN, whether through the nervous system or through an electrically-conductive signal path through the body cavity, the path including at least one of nerve tissue and other tissue. Tissue which is electrically conductive can affect nerves via conducted nerve signals, voltages, electric potentials, or other effects of applied signals, such as for example transdermally-applied signals.

A first electrode 202, shown located rostral to the person's thoracic spine, may be electrically coupled to the vagus nerve RVN transdermally, such that the nerve may be affected by signals, voltages, currents, and/or other electrical effects from the electrodes, passing current along the vagus nerve(s). A second electrode 204, located at a site caudal to the person's cervical spine as shown, may be coupled through the same side vagus nerve (via SN1, shown in FIG. 2) or alternatively (and in some embodiments, preferentially) through a portion of the first vagus nerve RVN, across the body, and to a site indicated as SN2. This arrangement is shown in FIG. 3.

A signal path between the electrodes 202, 204 may pass along one or more vagus nerves and/or other nerves, such as spinal nerves.

The electrodes may optionally include a third electrode 402 adjacent to or operatively coupled the second vagus nerve LVN of the human via transdermal coupling. The third electrode 402 may be located at a second location rostral to the human's thoracic spine, and may be deployed on an opposite side of the human relative to the first electrode 202, for example when arranged to stimulate the other vagus nerve. FIGS. 4 and 5 show a fourth electrode 404 electrically coupling to another nerve in electrical communication with the vagus nerves RVN and/or LVN, also via transdermal coupling, at a second site, to the human's cervical spine and optionally on a laterally opposite side relative to the second electrode 204.

FIG. 2 further depicts a waveform generator 208 coupled to the various electrodes 202, 204, and/or (if present) 402, 404 by electric wires or equivalent means (indicated by straight lines in the figure), which may be configured for applying a first therapeutic waveform to cause electric current flow between the first and second electrodes 202, 204 and along at least a portion of a vagus nerve (and/or for applying a therapeutic waveform of electric potential). The waveform generator 208 may further or alternatively be configured for applying a second therapeutic waveform to cause current or voltage flow between the third and fourth electrodes 402, 404 and along at least a portion of a vagus nerve or nerves RVN, LVN.

The output of the waveform generator 208 may be optionally controlled by a logic circuit 210 which may receive signals from a heart rate sensor coupled to the human; for example, as shown in FIG. 5, via electrodes 502, 504, into the logic circuit 210, such as an A/D convertor/amplifier channel pair portion of the logic circuit 210. The system 500 may detect heartbeats and their timing, and may calculate HRV (see FIG. 1, 118), exemplified as the variation of inter-beat intervals. As seen in FIG. 1, a decrease in HRV greater than a pre-determined threshold or ratio may cause the system 500 to stop treatment, in step 122. Optionally, after stopping treatment, (either for an HRV fault (step 120), or for a timeout (step 116)) identifying a scheduled treatment duration, the system 500 may generate a report, as seen in step 124. According to embodiments, “generating a report” may include the logic circuit 210 sending data corresponding to the completed treatment to a networked computer (not shown), wherein the networked computer generates the report, either alone or in combination with a networked application server.

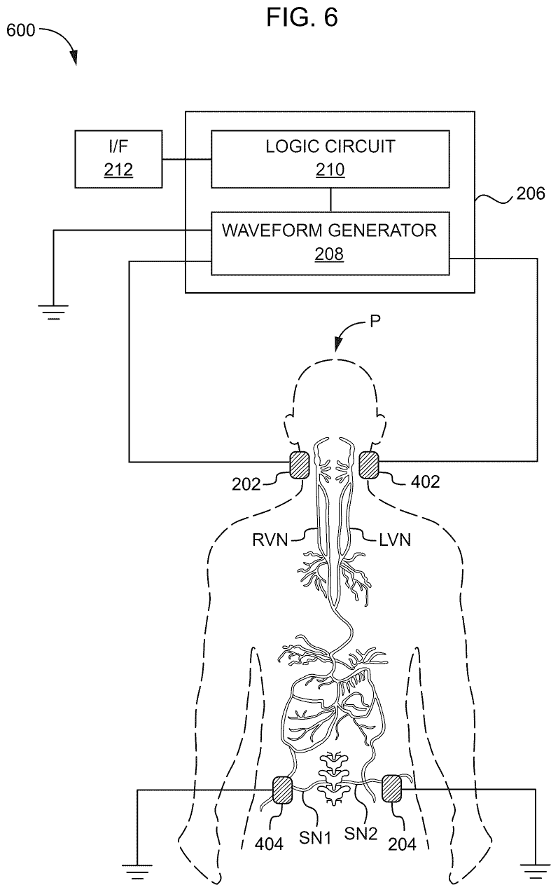

According to another embodiment, as shown in FIG. 6, the electrodes 204, 404 are electrically coupled to the vagus nerve(s) as described above with reference to FIG. 4, and to a circuit ground of the waveform generator 208, such that applying the therapeutic waveform, in step 114, includes applying the first and/or second waveform frequencies f₁, f₂, between the electrodes 202, 402, respectively, and circuit ground, at either or both of the electrodes 204, 404. A current path between electrode 202 and circuit ground (either or both of the electrodes 204, 404) may hereafter be referred to as current channel 1, while a current path between electrode 402 and circuit ground may hereafter be referred to as current channel 2.

As discussed below, there are a number of involuntary physiological parameters that may be indicative of an individual's mental and/or emotional state or health. These may include heart and breath rate, HRV, blood pressure, pupil dilation, skin temperature and conductivity, blood oxygenation, etc. Many of these parameters can be measured and/or monitored using equipment that is non-invasive and relatively simple to use, such that the equipment can be self-applied and used by a person with little or no training. Devices configured to detect and measure many of these parameters are well known, commercially available, and in common use. Many devices intended to be worn (e.g., on a wrist or head band), by athletes or other individuals incorporate sensors configured to detect and measure various of the parameters. Other devices are commonly carried by health professionals and used when examining patients, etc. Accordingly, the inventor contemplates standardized devices that can be operated by treatment subjects for self-administration without close supervision by trained practitioners.

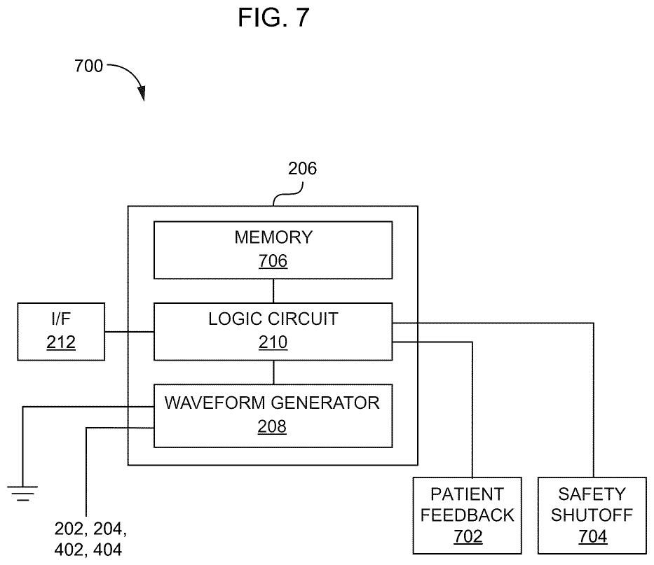

FIG. 7 is a diagram of a system 700 for activating an autonomic nervous system response, according to an embodiment, that is configured for self-administration of a therapeutic waveform schedule to the vagus nerve(s) of a therapy subject. The system 700 includes an electronic controller 206 and a user interface 212. The controller includes a waveform generator 208 and a logic circuit 210 as described above with reference to other embodiments. The controller 206 also includes signal and ground leads configured to be coupled to electrodes 202, 204, 402, 404, which a subject can self-apply in locations described above with reference to FIGS. 1-5.

The system 700 also includes a memory 706, an input for a signal from user feedback sensor 702 and an input for a signal from a safety shut-off switch 704. The memory 706 is configured to store programs for controlling administration of therapeutic waveform schedules by the logic circuit 210.

The user feedback sensor 702 is configured to monitor one or more of the physiological parameters described above, and the logic circuit 210 is configured to adjust the waveform schedule in response to detected changes of the physiological parameters. For example, in an embodiment in which the feedback sensor 702 is configured to monitor the subject's HRV, the logic circuit may respond to a change in HRV by modifying one or more aspects of the waveform, such as frequency, shape, strength, etc., depending upon the instructions stored in the memory 706 and the nature of the changes detected. Similarly, the logic may be configured to respond to changes in others of the parameters or combinations of parameters, depending upon the instructions stored in the memory 706 and the nature of the changes.

The safety shut-off switch 704 is configured to provide a shut-off signal in response to which the logic circuit is configured to terminate any waveform schedule in current operation. For example, according to one embodiment, the safety shut-off switch 704 includes a simple manually-operated switch that the subject can actuate in the event of discomfort or distress. If the logic circuit detects activation of the switch, it is configured to terminate the current waveform signal. According to another embodiment, the safety shut-off switch 704 includes an adjustment element, such as a knob or slider, etc., by which the subject can, at least to an extent, reduce the intensity of an applied current waveform in the event treatment becomes uncomfortable.

According to another embodiment, the safety shut-off switch 704 includes a sensor configured to detect a loss of consciousness of the subject. For example, in one embodiment, the subject is required to hold the switch 704 in one hand, and to maintain light pressure against a pressure pad. If the subject loses consciousness, such as by fainting, etc., the subject will release the pressure and the switch will send the shut-off signal.

According to an embodiment, the safety shut-off switch 704 may be incorporated as an element or component of the user interface 212.

During operation of the system 700, a practitioner stores a therapeutic waveform schedule in the memory 706 via the interface 212. The schedule may be tailored specifically for the intended subject or may be a standard schedule selected by the practitioner, depending upon the needs of the subject and the schedules that may be currently available. According to an embodiment, the memory 706 and/or the schedule(s) stored therein may be protected, e.g., by password, etc., to enable access by the practitioner but limit access by the treatment subject or other unauthorized individuals (beyond the degree of control necessary for self-administration of a therapeutic waveform schedule), to prevent modifications to the schedule that might prove harmful.

The subject applies electrodes 202, 204 and/or electrodes 402, 404 as shown and described with reference to FIGS. 2-5, and attaches leads to electrically couple the electrodes to the controller 206. Using the user interface 212, the subject selects the appropriate schedule and initiates application of the therapeutic waveform schedule, which is carried out by the logic circuit 210 in accordance with the instructions stored in the memory 706. The memory may be configured to save a record of the operation of the system, including dates and times of treatments, initial values of physical parameters detected by the feedback sensor, changes in those values, any modifications made by the control circuit, and ending values of the parameters. This enables the practitioner to review the subject's adherence to a prescribed treatment schedule as well as progress made during treatment.

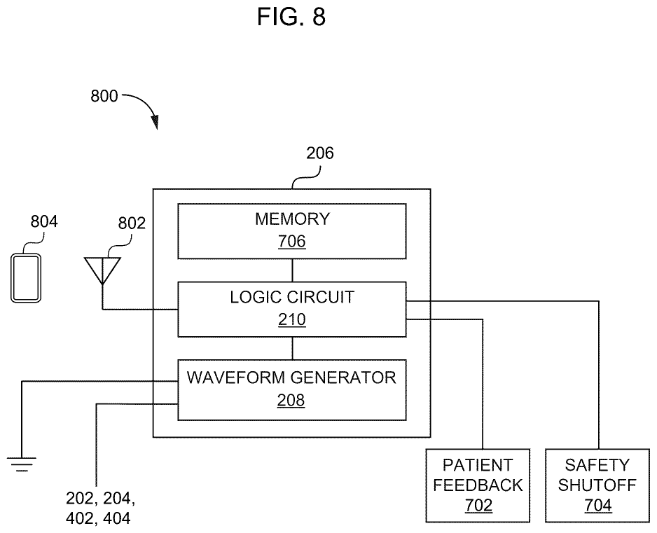

FIG. 8 is a diagram of a system 800 for activating an autonomic nervous system response, according to an embodiment. The system 800 is similar in many respects to the system 700 of FIG. 7, and it's operation is also similar. However, the system 800 also includes transmission and reception capabilities via an antenna 802. The antenna 802 may be configured to communicate via any appropriate wireless protocol, including Wifi, Bluetooth, cellphone signals, proprietary communication systems and/or protocols, etc. According to an embodiment, a treatment subject loads an “app” (application program) onto a cell phone or other remote device 804, which then performs some or all of the functions served, in other embodiments, by the interface 212. The app may also be configured to enable the practitioner to upload treatment schedules to the subject's remote device 804 and download results. The app may also be configured to protect the therapeutic waveform schedule from intentional or unintentional modification by the subject. In some embodiments, the phone 202 may also perform the functions served by the memory 706 in the embodiment of FIG. 7, which may in turn be omitted. In other embodiments, some or all of the functions are served by the onboard memory, with the phone mediating and serving only as the interface.

According to other embodiments, features of the embodiments of FIGS. 7 and 8 may be combined. For example, in one embodiment, the user interface 212 described with reference to FIG. 7 includes wireless communication capability, as described with reference to FIG. 8, such that a practitioner can upload instructions and/or download treatment results directly to the system without mediation by a remote device.

Referring to FIG. 8, according to an embodiment, an apparatus for applying a therapeutic current waveform to a human vagus nerve includes a waveform generator 208 configured to produce:
at a first terminal 202, a first therapeutic current waveform selected for application to a human subject via a first electrode transdermally coupled at a first location rostral to the subject's thoracic spine,
at a second terminal 402, a second therapeutic current waveform selected for application to the human subject via a second electrode transdermally coupled at a second location rostral to the subject's thoracic spine, and
at a third terminal 204, 404, a ground potential configured to be electrically coupled to one or more electrodes transdermally coupled at respective locations caudal to the subject's cervical spine. The apparatus also includes a logic circuit 210 configured to control operation of the waveform generator.

The logic circuit 210 may include a feedback input configured to receive a feedback signal from a sensor 702 coupled to the subject, the logic circuit 210 being configured to regulate operation of the waveform generator 208 in response to the feedback signal.

Referring to FIGS. 2 through 8, according to an embodiment, an apparatus for applying a therapeutic current waveform to a human vagus nerve includes a housing 206, a logic circuit 210 disposed in the housing 206, and a waveform generator 208 operatively coupled to the logic circuit 210 and disposed in the housing 206, the waveform generator 208 being configured to generate a therapeutic current waveform responsive to the logic circuit 210. One or more electrode connectors (not shown) may be disposed in a wall of the housing 206 and operatively coupled to the waveform generator 208, the one or more electrode connectors being configured to couple to adhesive electrodes 202, 204, the adhesive electrodes 202, 204 being configured for coupling to respective locations on human skin for transdermally applying the therapeutic current waveform along at least a first vagus nerve of the human. The logic circuit 210 may be configured to control the waveform generator 208 to generate a sequence of different frequency therapeutic current waveforms selected to cause a parasympathetic response in an autonomic nervous system of the human.

The apparatus may further include at least one sensor interface operatively coupled to the logic circuit 210, the at least one sensor interface being configured to couple to one or more sensors 702 configured to sense a detectable human response to the sequence of different frequency therapeutic current waveforms. The logic circuit 210 may be configured to cause a modification of the therapeutic current waveform or the sequence of different frequency therapeutic current waveforms responsive to the sensed detectable human response.

Responsive to the sensed human response, the logic circuit 210 may be configured to control a duration of the therapeutic current waveform and/or the sequence of different frequency therapeutic current waveforms. Additionally or alternatively, the logic circuit 210 may be configured to control an electrical current of the therapeutic current waveform and/or the sequence of different frequency therapeutic current waveforms. Additionally or alternatively, the logic circuit 210 may be configured to control a frequency of the therapeutic current waveform or the sequence of different frequency therapeutic current waveforms.

The at least one sensor interface may be configured to operatively couple to a sensor 702 configured to measure pupil diameter, heart rate, blood pressure, HRV, and/or an electroencephalogram (EEG).

The apparatus may further include an electronic display (not shown) or electronic display interface operatively coupled to the logic circuit 210. The logic circuit may be configured to cause electronic display of an indication of active status corresponding to active application of the therapeutic current waveform, an indication of inactive status corresponding to not applying the therapeutic current waveform, elapsed time of application of the therapeutic current waveform, countdown time to the end of the application of the therapeutic current waveform, frequency of the therapeutic current waveform, a sensed value corresponding to parasympathetic state, and/or current output by the waveform generator, for example.

Referring most specifically to FIG. 8, the apparatus may include a wireless interface 802 operatively coupled to the logic circuit 210, the wireless interface 802 being configured to connect to a smart phone 804. A smart phone software application may be configured to provide control input to the logic circuit 210. In an embodiment, the smart phone software application is configured to operate a front camera to monitor changes in pupil size of the human receiving the therapeutic current waveform via the front camera, and to transmit control signals to the logic circuit 210 via the wireless interface 802 responsive to the changes in pupil size. Additionally or alternatively, the smart phone software application may be configured to operate the front camera to monitor a state of alertness of the human receiving the therapeutic current waveform, and to transmit control signals to the logic circuit 210 via the wireless interface 802 responsive to the changes in alertness.

The smart phone software application may be configured to display a progress icon on an electronic display of the smart phone, the progress icon corresponding to an autonomic state of the human and/or a progress of a therapeutic current session.

The smart phone software application may be configured to collect a therapeutic current waveform application and a sensed response to the therapeutic current waveform application and to upload the therapeutic current waveform application and the sensed response to a networked supervisory server computer (not shown).

The smart phone software application may be operative to receive a command or commands from the human user related to a vagus nerve therapeutic current stimulation session, such as to reduce a current level of the therapeutic current waveform.

Referring again to FIGS. 2 through 8, according to embodiments, an apparatus for applying a therapeutic current waveform to a human vagus nerve includes a housing 206, a logic circuit 210 disposed in the housing 206, and a waveform generator 208 operatively coupled to the logic circuit 210 and disposed in the housing 206, the waveform generator 208 being configured to generate a therapeutic current waveform responsive to the logic circuit 210. One or more electrode connectors may be disposed in a wall of the housing 206 and operatively coupled to the waveform generator 208, the one or more electrode connectors being configured to couple to adhesive electrodes 202, 204, 402, 404, the adhesive electrodes 202, 204, 402, 404 being configured for coupling to respective locations on human skin for transdermally applying the therapeutic current waveform along first and second vagus nerves of the human. The logic circuit 210 may be configured to control the waveform generator 208 to generate a first therapeutic current waveform coupled at least through the adhesive electrode 202 disposed on a first lateral side of the human rostral to the human's thoracic spine and to generate a second therapeutic current waveform, different from the first therapeutic current waveform, coupled at least through the adhesive electrode 402 disposed on a second lateral side of the human opposite to the first lateral side of the human rostral to the human's thoracic spine, to one or more adhesive electrodes 204, 404 disposed caudal to the human's cervical spine. The first and second waveforms may be characterized by different frequencies that are applied simultaneously.

Referring to FIG. 1, according to an embodiment, a method for operating a vagus nerve therapeutic current stimulation apparatus configured to activate an autonomic nervous system response in a human includes applying a therapeutic current waveform between a first electrode transdermally coupled at a location rostral to a human's thoracic spine and a second electrode transdermally coupled at a location caudal to the human's cervical spine and controlling the therapeutic current waveform to have a peak current of between 100 and 300 microamps. In step 118, a signal is received from a sensor. When the signal is within a safe range, the method may loop again to step 114, where step 114 includes regulating one or more characteristics of the therapeutic current waveform in response to the signal from the sensor.

Receiving a signal from a sensor may include receiving a signal from a pupilometer. Additionally or alternatively, receiving a signal from a sensor may include receiving a signal from a heartbeat sensor. The method may further include continuously calculating a heartrate variability on the basis of the received signal. Regulating one or more characteristics of the therapeutic current waveform in response to the signal from the sensor may include regulating the one or more characteristics in response to changes in the continuously calculated heartrate variability.

In some embodiments, receiving a signal from a sensor includes receiving a signal from an electroencephalograph, and regulating one or more characteristics of the therapeutic current waveform in response to the signal from the sensor includes regulating the one or more characteristics in response to changes to one or wave components of the signal from the electroencephalograph, including at least one of Theta, Delta, Alpha, Beta, and Gamma waves.

Applying a therapeutic current waveform between a first electrode and a second electrode in step 114 may include applying the therapeutic current waveform between the first electrode 202 and either or both of the second electrode 204 and a third electrode 404 transdermally coupled at a second location caudal to the human's cervical spine (e.g., see FIGS. 4-6). Applying the therapeutic current waveform between the first electrode 202 and either or both of the second electrode 204 and a third electrode 404 may include applying a first therapeutic current waveform between the first electrode 202 and either or both of the second electrode 204 and the third electrode 404 while simultaneously applying a second therapeutic current waveform between a fourth electrode 402 transdermally coupled at a different location rostral to the human's thoracic spine and either or both of the second electrode 402 and the third electrode 404.

Regulating one or more characteristics of the therapeutic current waveform in response to the signal from the sensor, in step 114, may include regulating one or more of a peak amplitude, a duty cycle, a duration, and/or a wave shape of the waveform.

According to an embodiment, a method for operating a vagus nerve therapeutic current stimulation apparatus configured to activate an autonomic nervous system response in a human includes, in step 102, electrically coupling a first electrode 202 to a human subject at a location rostral to the subject's thoracic spine, and in step 104, electrically coupling a second electrode 204 to the human subject at a location caudal to the subject's cervical spine. Step 114 includes applying a therapeutic current waveform between the first electrode 202 and the second electrode 204 while controlling the therapeutic current waveform to have a peak current of between 100 and 300 microamps. Step 118 includes receiving a signal from a sensor, and looping back to step 114, regulating one or more characteristics of the therapeutic current waveform in response to the signal from the sensor.

According to an embodiment, a method for operating a vagus nerve therapeutic current stimulation apparatus configured to activate an autonomic nervous system response in a human, includes measuring an impedance between first and second electrodes 202, 204 with a therapeutic current generation circuit, the impedance being consistent with electrically coupling the first electrode to a first vagus nerve via a transdermal coupling at a location rostral to the human's thoracic spine and electrically coupling the second electrode to a nerve in electrical communication with the first vagus nerve or a second vagus nerve, via a transdermal coupling at a site caudal to the human's cervical spine. A control input is received into a control circuit operatively coupled to the therapeutic current generation circuit, the control input being indicative that a therapeutic current waveform is to be applied between the first and second electrodes 202, 204. Responsive to the control input, the method includes generating the therapeutic current waveform to cause current flow between the first and second electrodes 202, 204 along and through at least a portion of the first vagus nerve or first and second vagus nerves between the first and second electrodes 202, 204.

Generating the therapeutic current waveform may include generating a series of voltage spikes. For example, generating the therapeutic current waveform may include generating a series of alternating polarity constant current voltage spikes having a peak current between 100 and 300 microamps. In a particular example, generating the therapeutic current waveform includes generating a series of alternating polarity constant current voltage spikes each having a peak current of about 200 microamps. Generating the therapeutic current waveform may include generating a waveform including alternating polarity voltage spikes having a duty cycle below 5%.

In another embodiment, generating the therapeutic current waveform in step 114 includes generating a square wave waveform. For example, generating the square wave waveform may include generating a series of monophasic square waves. In another example, generating the therapeutic current waveform includes generating a hybrid waveform including a sharp rising edge, a constant voltage portion, and an exponential decay portion. The sharp rising edge may include overshoot.

To accommodate individuals for whom the therapeutic current causes discomfort, the user may actuate a user interface operatively coupled to the control circuit to reduce the peak current delivered by the therapeutic current generation circuit. Accordingly, the method may further include receiving, into the control circuit, a command to reduce a peak current output by the therapeutic current waveform generator and responsively reducing the peak current output by the therapeutic current generation circuit to produce a reduced peak current.

According to embodiments, the waveform current is between 100 and 300 microamps.

Referring to FIG. 2, the first and second electrodes 202, 204 may be operatively coupled to vagus nerves on the same lateral side of the human body to cause the therapeutic current to pass along substantially the first vagus nerve. Referring to FIG. 3, the first and second electrodes 202, 204 may be operatively coupled to vagus nerves on laterally opposite sides of the human body to cause the therapeutic current to pass along and between the first and second vagus nerves.

Referring to FIGS. 4-6, the method may include generating a second therapeutic current waveform to cause current flow between third and fourth electrodes 402, 404 transdermally coupled to first or first and second vagus nerves on laterally opposite sides of the human body respectively opposite to the first and second electrodes 202, 204.

Generating the therapeutic current waveform may include generating first and second therapeutic current waveforms respectively coupled to the first and second electrodes 202, 204 and to the third and fourth electrodes 402, 404. According to embodiments, generating the first and second therapeutic current waveforms includes generating a pair of waveforms having respective different frequencies.

According to embodiments, generating the first and second therapeutic current waveforms includes generating a sequence of pairs of waveforms, wherein at least one of the frequencies of the pair of frequencies changes between successive ones of the sequence of pairs of waveforms. For example, generating the therapeutic current waveform may include generating a plurality of therapeutic current waveforms having different frequencies and applying the plurality therapeutic current waveforms as a battery of waveforms in a sequence.

The method may include receiving a sensor value into the control circuit from a sensor configured to read an observable characteristic of the human, and with the control circuit, verifying that the sensor value meets a predetermined human characteristic criterion. The method may include receiving a sensor value into the control circuit from a sensor configured to read an observable characteristic of the human, and with the control circuit, modifying a current or frequency of the therapeutic current waveform responsive to the sensor value. The method may include receiving a sensor value into the control circuit from a sensor configured to read an observable characteristic of the human, and with the control circuit, modifying duration of application of the therapeutic current waveform responsive to the sensor value.

In an embodiment, the method may include receiving a user input via an interface into a logic circuit, the user input corresponding to a condition to be treated and/or a therapeutic waveform schedule. For example, receiving the therapeutic waveform schedule into the logic circuit may include receiving a waveform frequency. In an embodiment, receiving the therapeutic waveform schedule into the logic circuit includes receiving two waveform frequencies; and the method may further include loading corresponding parameters into the waveform generator to cause the waveform generator to cooperate with four electrodes 202, 204, 402, 404 electrically coupled to the vagus nerve to apply a first push-pull waveform frequency to the first pair of electrodes 202, 204 through portions of two respective vagus nerves, and to apply a second push-pull waveform frequency to the second pair of electrodes 402, 404; where the four electrodes are disposed as cross-body pairs including the first pair 202, 204 and a second pair 402, 404, as shown in FIGS. 4 and 5. Each of two frequencies may be determined responsive to the condition to be treated or the therapeutic waveform schedule received into the logic circuit from the interface.

### EXAMPLES

### Test Protocol

Single-blind testing was performed with a recruited test group of 22 healthy subjects using an electrode set-up corresponding to that shown in FIG. 6. The electrodes were conventional pressure sensitive conductive adhesive electrodes.

Each test subject wore a Fitbit Sense for a week in which heart rate was measured once per minute and heart rate variability (HRV), expressed as RMSSD (root-mean-square of successive differences), was measured once every five minutes during deep sleep. Following a week of monitoring, a battery of tests including application of therapeutic current frequencies shown in Table 1, was administered sequentially. Each subject was again monitored for one week following the application of the therapeutic current battery.

The stimulation equipment was a commercially-available frequency-specific therapeutic current stimulator, model CustomCARE FSM III, available from Mittag Holistic Chiropractic PA, of Minnetonka, Minnesota U.S.A.

A test battery was designed to include a sequence of therapeutic current pulses at frequencies shown below, intended to cause an increase in neural tone of the autonomic nervous system, corresponding to a state generally associated with a parasympathetic response.

TABLE 1

| Seq | Waveshape | Freq1 | Freq2 | Current | Duration |
|---|---|---|---|---|---|
| 1 | Square | 40.0 | 89.0 | 200 | 4 m |
| 2 | Square | 40.0 | 94.0 | 200 | 4 m |
| 3 | Square | 94.0 | 109.0 | 200 | 4 m |
| 4 | Square | 81.0 | 109.0 | 200 | 4 m |
| 5 | Square | 49.0 | 109.0 | 200 | 4 m |

For the purpose of determining whether a current applied would likely be perceptible and/or uncomfortable, a test subject, not included in the 22 test subjects referred to above and hereafter, received therapeutic current (or not) through electrodes arranged according to the arrangement shown in FIG. 6. The test subject recorded his qualitative feeling and indicated current observations as shown in Table 2. In Table 2, “Current channel 1” refers to a representative waveform signal applied to an electrode corresponding to the electrode 202 of FIG. 6, while “Current channel 2” refers to a representative waveform signal applied to an electrode corresponding to the electrode 402 of FIG. 6. The sensation reported by the subject was tingling consistent with transmission of current, in microamps, indicated by the test equipment.

TABLE 2

| Electrodes Connected | | | | |
|---|---|---|---|---|
| 202 | 204 | 402 | 404 | Observations |
| | Yes | | Yes | No current or sensation |
| Yes | | Yes | | No current or sensation |
| | | Yes | Yes | Current channel 2 and sensation |
| Yes | Yes | | | Current channel 1 and sensation |
| Yes | | | Yes | Current channel 1 and sensation |
| | Yes | | Yes | Current channel 2 and sensation |
| Yes | Yes | | Yes | Current channel 1 and more sensation |
| | Yes | Yes | Yes | Current channel 2 and more sensation |
| Yes | | Yes | Yes | Current both channels and sensation |
| Yes | Yes | Yes | | Current both channels and sensation |
| Yes | Yes | Yes | Yes | Current both channels and sensation |

Referring again to Table 1, for each sequential test in the test battery, a monophasic square wave at respective indicated frequencies and having approximately a 50% duty cycle was administered to each of two rostral electrodes 202, 402, while two caudal electrodes 204, 404 were held at ground. This arrangement is shown in FIG. 6. Each test sequence included the waveform at a first frequency, indicated as “Freq1”, being applied to electrode 202; while at the same time a waveform at a second frequency, indicated as “Freq2”, was applied to electrode 402. Each waveform pair was applied for four minutes (indicated as 4 m), and then the test battery proceeded to the next frequency pair in the sequence, through a 20-minute period during which each of the five frequency combinations shown in Table 1 was applied sequentially.

Voltage was set to initially produce an indicated 200 microamps current reported on a display of the test apparatus. The waveform used in the tests was a square wave at about a 50% duty cycle that was not controlled for constant current. If a particular test subject found the applied voltage uncomfortable, the test subject was allowed to adjust the voltage downward in decrements corresponding to approximately 20 milliamps current, until tolerable. The resultant current applied to each test subject is shown in Table 3.

TABLE 3

| | Indicated Stimulation |
|---|---|
| | Current |
| SUBJECT | (microamps) |
| 1 | 200 |
| 2 | 200 |
| 3 | 200 |
| 4 | 200 |
| 5 | 180 |
| 6 | 180 |
| 7 | 20 |
| 8 | 200 |
| 9 | 200 |
| 10 | 200 |
| 11 | 200 |
| 12 | 200 |
| 13 | 200 |
| 14 | 200 |
| 15 | 200 |
| 16 | 200 |
| 17 | 200 |
| 18 | 200 |
| 19 | 200 |
| 20 | 200 |
| 21 | 200 |
| 22 | 200 |

Wires from the test apparatus were attached to respective adhesive patch electrodes disposed on the subject as shown in FIG. 6. Each pair of patch electrodes were coupled to lateral opposite sides of the test subjects as shown in FIG. 6 to produce, during any one sequential portion of the test battery, a pair of dissimilar waveforms input in a rostral position arranged as shown, with corresponding ground electrodes coupled at caudal positions as shown.

Frequencies of the test battery were selected and intended to induce a parasympathetic vagus tone effect.

Current was initially set to 200 microamps, as reported by a display on the test equipment. Subjects were advised to cause a reduction of voltage (corresponding to a reduction in current) if desired to improve comfort.

Three of twenty-two test subjects, subjects 5, 6, and 7, adjusted the current downward during the test batteries, understood to be for reasons of comfort. One test subject, subject 7, manually decreased current to the lowest constant current among the test subjects, as shown in Table 3. Another test subject, number 12, did not complete the test battery due to syncope, and dropped out.

### Results

The test battery was found to cause a plurality of detectable effects, which may point to plural modalities of use across diagnostic and therapeutic genera related to functional aspects of vagus-coupled animal, and particularly human systems. At least two detectable effects suggest strategies described below.

During the entire testing session, autonomic state-indicative and neural signals were collected. The monitored signals included respiration, electro-cardiogram (ECG), non-invasive beat-to-beat blood pressure, electrodermal activity, skin temperature, all with equipment available from ADinstruments, of Dunadin, New Zealand. The monitored signals also included electroencephalogram (EEG) using a model DSI-24 “dry sensor interface”, available from Wearable Sensing, of San Diego, California USA. The monitored signals also included pupillometry, measured by an eye tracker model Tobii Glasses Pro 2, discontinued but originally available from Tobii of Stockholm, Sweden.

Three detected effects commonly associated with high autonomic tone, i.e., parasympathetic as compared to sympathetic state function are reported. Table 4 summarizes significant responses by detection modality for the test population.

TABLE 4

| Detection Modality | Mean Response |
|---|---|
| Pupilometry | Decreased diameter |
| | 4.25 mm » 3.75 mm |
| Heartrate Variability | Increase |
| Electro Encephalogram | Increase in frontal theta power |
| | Decrease in frontal gamma power |

### Pupillometry

It is generally understood that pupil diameter constricts as the nervous system moves into a parasympathetic state. Similarly, when in a parasympathetic state, humans are known to generally experience decrease in heart rate, blood pressure, and adrenalin.

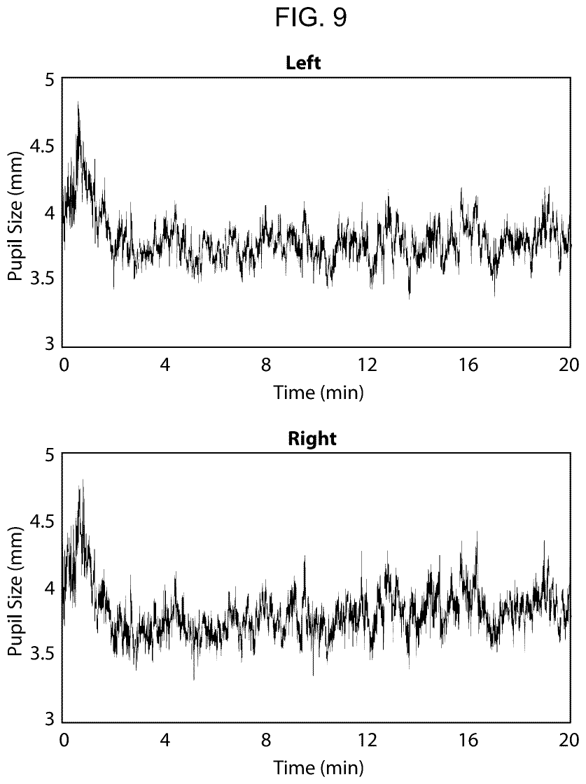

The mean response of the test subjects who completed the battery, as shown in FIG. 9, was an initial increase in pupil diameter (in both eyes) from 4 millimeters to 4.25 millimeters over the first minute of the first frequency pair in the sequence of the battery, followed by a decrease in pupil diameter from 4.25 millimeters to about 3.75 millimeters over the second minute. Between 2 minutes and 20 minutes (20 minutes being the duration of the entire test battery), mean pupil diameter remained substantially constant at about 3.75 millimeters.

The inventors interpreted this result as being indicative that the test battery had a detectable and statistically significant effect on inducing a parasympathetic response across the test subjects. A responsive group of test subjects indicated greater parasympathetic response and reduced variance compared to the non-responsive group and to the entire population.

### Heart Rate Variability

A relatively high heartrate variability (HRV), as measured using a root-mean-square of successive differences (RMSSD), is generally considered to be a positive indicator of health compared to lower HRV. This may be interpreted as a higher HRV indicating a nervous system capable of more effectively responding to demands on the cardiovascular system.

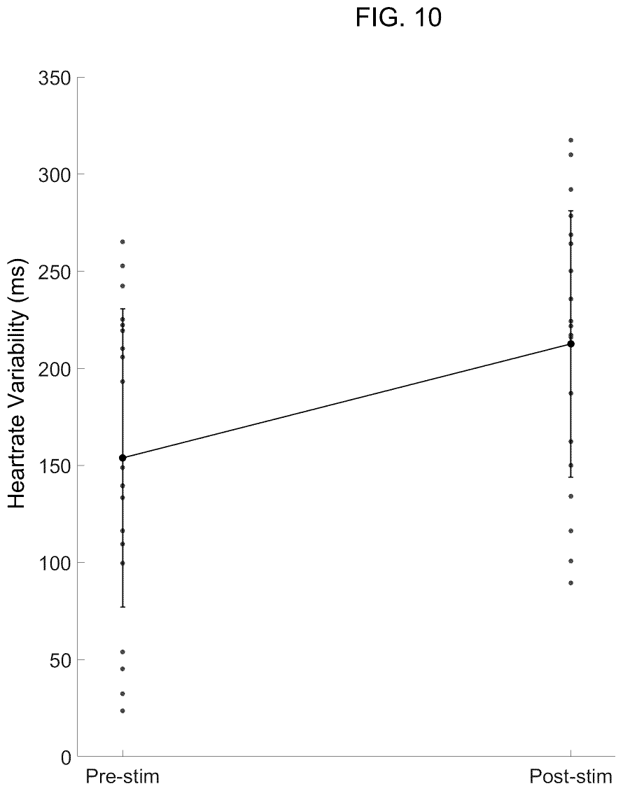

FIG. 10 is a chart showing measured responses of HRV to the test battery for all subjects.

Test subjects were divided into responsive and non-responsive groups.

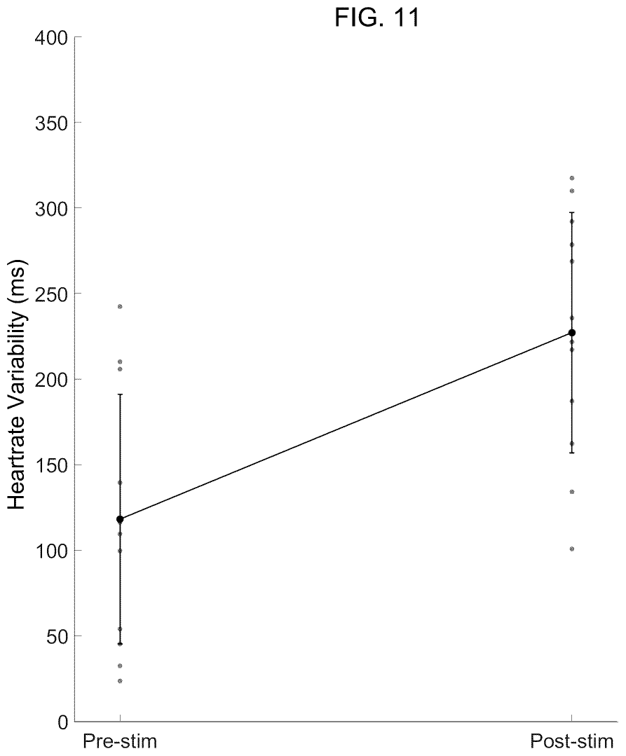

FIG. 11 is a chart showing measured responses of HRV to the test battery for responsive group subjects.

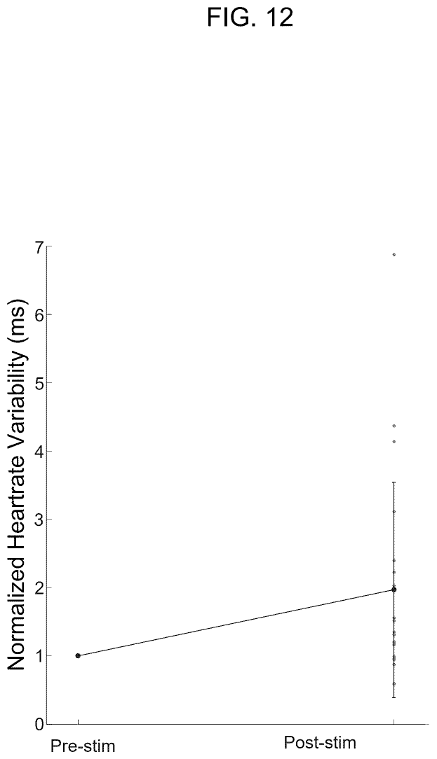

FIG. 12 is a chart showing normalized responses of HRV to the test battery for all subjects.

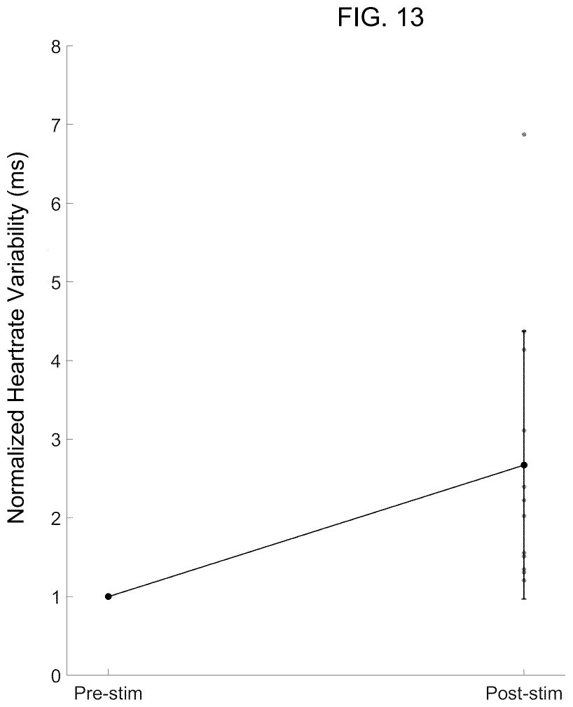

FIG. 13 is a chart showing normalized responses of HRV to the test battery for responsive group subjects.

Subject response generally exhibited a significant increase in HRV, comparing before and after the test battery. This increase amounted to nearly a doubling of HRV over the 21 subjects who completed the test battery and a 150% increase among responders.

### EEG

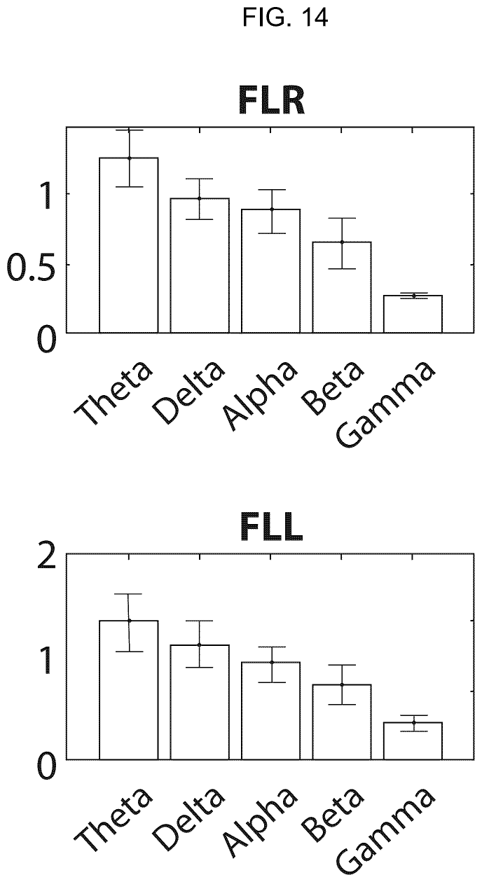

FIG. 14 is a set of charts showing normalized changes in EEG passband power measured for frontal right (FLR) and frontal left (FLL) lobe positions.

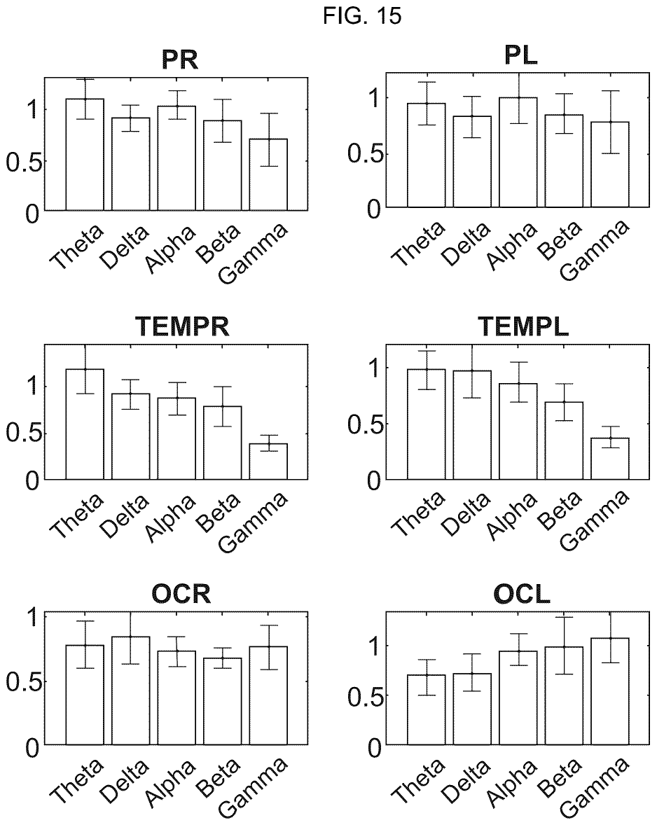

FIG. 15 is a set of charts showing normalized changes in EEG passband power measured for parietal (P), temporal (TEMP), and occipital (OC) lobe positions, with separate graphs for left (L) and right (R) respective lobes.

Referring to FIG. 14 especially, EEG data as frontal lobe activity exhibited a dramatic change in response to the test battery. Mean normalized delta power, before and after the therapeutic current test battery treatment, remained about constant. Delta frequencies, 0 Hz to 4 Hz, may typically be associated with sleep.

Alpha power decreased a small amount, less than 10% on mean, to somewhat greater than 0.9, normalized. Alpha frequencies tend to be associated with present awareness and alertness, without or without being in an aroused (sympathetic) state.

Beta power, on average, decreased to about 0.7.

Theta power increased significantly, by about 20 to 30 percent (1.2 to 1.3) after the test battery. Theta corresponds to frequency components of 4 Hz to 8 Hz that may be typically associated with activities such as meditation and prayer.

Gamma power decreased dramatically, to approximately 0.25 (about 75 percent reduction), normalized. Gamma activity, 40 Hz to 80 Hz, has typically been associated with agitation, in psychiatric studies.

The EEG response was bilateral across both frontal lobes, and had relatively small variance across the population, the gamma variance being the smallest, while also showing the greatest response.

The inventors believe this result suggests a response that compares favorably to administration of anti-anxiety medications intended to reduce behaviors found corresponding to high gamma. One might conclude that the Model battery, as indicated by EEG results, created a brain function similar to a meditative state with very low agitation across the study population.

Clinical use cases may range from cardiac medicine to neurologic and psychiatric use cases. For example, the subject non-invasive vagus electrical stimulation may prove valuable for indications ranging from treatment of drug-resistant epilepsy, sub-acute stroke rehabilitation augmentation, and treatment of post-traumatic stress disorder (PTSD), for which, to the inventor's knowledge, only implanted electrical stimulators have been reported in previous studies.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

What is claimed is:

1. An apparatus for applying a therapeutic current waveform to a human vagus nerve, comprising:
a housing;
a waveform generator disposed in the housing and configured to produce:
at a first terminal, a first therapeutic current waveform selected for application to a human subject via a first electrode transdermally coupled at a first location rostral to the subject's thoracic spine, and
at a third terminal, a ground potential configured to be electrically coupled to one or more electrodes transdermally coupled at respective locations caudal to the subject's cervical spine; and
a logic circuit disposed in the housing, operatively coupled to the waveform generator and configured to control operation of the waveform generator.

2. The apparatus of claim 1 wherein the logic circuit includes a feedback input configured to receive a feedback signal from a sensor coupled to the subject, the control circuit configured to regulate operation of the waveform generator in response to the feedback signal.

3. The apparatus of claim 2, wherein the logic circuit is configured to receive, via the feedback input, a feedback signal from a pupilometer, and to regulate operation of the waveform generator in response to changes in the feedback signal.

4. The apparatus of claim 2, wherein the logic circuit is configured to receive, via the feedback input, a feedback signal from a cardiac function sensor, to continuously calculate a heartrate variability on the basis of the feedback signal, and to regulate operation of the waveform generator in response to changes in the continuously calculated heartrate variability.

5. The apparatus of claim 2, wherein the logic circuit is configured to receive, via the feedback input, a feedback signal from an electroencephalograph, and to regulate operation of the waveform generator in response to changes to one or more wave components of the signal from the electroencephalograph, including at least one of Theta, Delta, Alpha, Beta, and Gamma waves.

6. The apparatus of claim 2, comprising:
a computer memory operatively coupled to the logic circuit and configured to store instructions for application of therapeutic current waveforms; and
a user interface operatively coupled to the logic circuit and configured to enable a user to select a therapeutic waveform schedule from the memory and to control the logic circuit to initiate and/or terminate operation of a selected therapeutic waveform schedule.

7. The apparatus of claim 6, comprising:
a safety shut-off switch operatively coupled to the logic circuit, configured to provide a shut-off signal; and
wherein the logic circuit is configured to terminate operation of the waveform generator upon receipt of the shut-off signal.

8. The apparatus of claim 2, further comprising:
an antenna operatively coupled to the logic circuit;
wherein the logic circuit is configured to communicate wirelessly with a user interface device via at least one of the group consisting of Bluetooth, WiFi, and a cellular signal.

9. (canceled)

10. The apparatus of claim 8, comprising:
a smart phone including a software application configured to enable the smart phone to function as the user interface device and to download instruction data from a practitioner, to transmit the instruction data to the memory via the logic circuit, to initiate and terminate a therapeutic waveform schedule stored in the memory; and to upload data from the memory to the practitioner.

11. The apparatus of claim 1, further comprising:
one or more electrode connectors disposed in a wall of the housing and operatively coupled to the waveform generator, the one or more electrode connectors being configured to couple to adhesive electrodes, the adhesive electrodes being configured for coupling to respective locations on human skin for transdermally applying the therapeutic current waveform along at least a first vagus nerve of the human;
wherein the logic circuit is configured to control the waveform generator to generate a sequence of different frequency therapeutic current waveforms selected to cause a parasympathetic response in an autonomic nervous system of the human.

12. The apparatus of claim 11, further comprising:
at least one sensor interface operatively coupled to the logic circuit, at least one sensor interface being configured to couple to one or more sensors configured to sense a detectable human response to the sequence of different frequency therapeutic current waveforms;
wherein the logic circuit is configured to cause a modification of the therapeutic current waveform or the sequence of different frequency therapeutic current waveforms responsive to the sensed detectable human response.

13. The apparatus of claim 12, wherein the logic circuit is configured to change at least one selected from the group consisting of a duration of the therapeutic current waveform, a duration of the sequence of different frequency therapeutic current waveforms, an electrical current of the therapeutic current waveform, and the frequency of the therapeutic current waveform.

14. The apparatus of claim 12, wherein the at least one sensor interface is configured to operatively couple to a sensor configured to measure at least one selected from the group consisting of pupil diameter, heart rate, blood pressure, heart rate variability (HRV), and an electroencephalogram (EEG).

15. The apparatus of claim 11, further comprising:
an electronic display or electronic display interface operatively coupled to the logic circuit, the logic circuit being configured to cause electronic display of at least one selected from the group consisting of an indication of active status corresponding to active application of the therapeutic current waveform, an indication of inactive status corresponding to not applying the therapeutic current waveform, elapsed time of application of the therapeutic current waveform, countdown time to the end of the application of the therapeutic current waveform, the frequency of the therapeutic current waveform, a sensed value corresponding to parasympathetic state, and current output by the waveform generator.

16. The apparatus of claim 11, further comprising:
a wireless interface operatively coupled to the logic circuit, the wireless interface being configured to connect to a smart phone; and
a smart phone software application configured to provide control input to the logic circuit.

17. The apparatus of claim 16, wherein the smart phone software application is configured to operate a front camera to monitor changes in pupil size of the human receiving the therapeutic current waveform, and to transmit control signals to the logic circuit via the wireless interface responsive to the monitored changes in pupil size.

18. The apparatus of claim 16, wherein the smart phone software application is configured to operate a front camera to monitor a state of alertness of the human receiving the therapeutic current waveform, and to transmit control signals to the logic circuit via the wireless interface responsive to the monitored changes in alertness.

19. The apparatus of claim 16, wherein the smart phone software application is configured to display a progress icon on an electronic display of the smart phone, the progress icon corresponding to at least one selected from the group consisting of an inferred autonomic state of the human and a temporal progress of a therapeutic current session.

20. The apparatus of claim 16, wherein the smart phone software application is configured to receive information about therapeutic current waveform and a sensed response to the therapeutic current waveform, and to upload the therapeutic current waveform information and the sensed response to a networked supervisory server computer.

21. The apparatus of claim 16, wherein the smartphone software application is configured to receive a command from the human to reduce a current level of the therapeutic current waveform.

22. The apparatus of claim 1:
wherein the waveform generator is further configured to produce, at a second terminal, a second therapeutic current waveform selected for application to the human subject via a second electrode transdermally coupled at a second location rostral to the subject's thoracic spine; and
further comprising:
one or more electrode connectors disposed in a wall of the housing and operatively coupled to the waveform generator, the one or more electrode connectors being configured to operatively couple to adhesive electrodes, the adhesive electrodes being configured for coupling to respective locations on human skin for transdermally applying the therapeutic current waveform along first and second vagus nerves of the human;
wherein the logic circuit is configured to control the waveform generator:
to generate a first therapeutic current waveform, operatively coupled to the first terminal, and coupled at least through the adhesive electrode disposed on a first lateral side of the human rostral to the human's thoracic spine, and
to generate a second therapeutic current waveform, different from the first therapeutic current waveform, operatively coupled to the second terminal, and coupled at least through the adhesive electrode disposed on a second lateral side of the human opposite to the first lateral side of the human rostral to the human's thoracic spine; and
further comprising:
one or more adhesive electrodes, operatively coupled to the third terminal, disposed caudal to the human's cervical spine.

23. The apparatus of claim 22, wherein the first and second waveforms are characterized by different frequencies that are applied simultaneously.

24. A method for operating a vagus nerve therapeutic current stimulation apparatus configured to activate an autonomic nervous system response in a human, comprising:
applying a therapeutic current waveform between a first electrode transdermally coupled at a location rostral to a human's thoracic spine and a second electrode transdermally coupled at a location caudal to the human's cervical spine;
controlling the therapeutic current waveform to have a peak current of between 100 and 300 microamps;
receiving a signal from a sensor operatively coupled to the human; and
regulating one or more characteristics of the therapeutic current waveform in response to the signal from the sensor.

25. The method of claim 24 wherein the receiving the signal from the sensor comprises receiving the signal from a pupilometer.

26. The method of claim 24 wherein receiving the signal from the sensor includes receiving the signal from a heartbeat sensor, the method further comprising:
continuously calculating a heartrate variability on the basis of the received signal;
wherein regulating the one or more characteristics of the therapeutic current waveform in response to the signal from the sensor includes regulating the one or more characteristics in response to changes in the continuously calculated heartrate variability.

27. The method of claim 24:
wherein receiving the signal from the sensor comprises receiving the signal from an electroencephalograph; and
wherein regulating the one or more characteristics of the therapeutic current waveform in response to the signal from the sensor includes regulating the one or more characteristics in response to changes to one or more wave components of the signal from the electroencephalograph, including at least one of Theta, Delta, Alpha, Beta, and Gamma waves.

28. The method of claim 24 wherein:
applying the therapeutic current waveform between the first electrode and the second electrode includes applying the therapeutic current waveform between the first electrode and either or both of the second electrode and a third electrode transdermally coupled at a second location caudal to the human's cervical spine.

29. The method of claim 28, further comprising:
simultaneous with applying the first therapeutic current waveform, applying a second therapeutic current waveform between a fourth electrode transdermally coupled at a different location rostral to the human's thoracic spine and either or both of the second electrode and the third electrode.

30-35. (canceled)

36. The method of claim 24, further comprising:
measuring an impedance between first and second electrodes with a therapeutic current generation circuit, the impedance being consistent with electrically coupling the first electrode to a first vagus nerve via a transdermal coupling at a location rostral to the human's thoracic spine and electrically coupling the second electrode to a nerve in electrical communication with the first vagus nerve or a second vagus nerve, via a transdermal coupling at a site caudal to the human's cervical spine;
receiving a control input into a control circuit operatively coupled to the therapeutic current generation circuit, the control input being indicative that a therapeutic current waveform is to be applied between the first and second electrodes; and
generating the therapeutic current waveform to cause current flow between the first and second electrodes.

37. The method of claim 24, wherein generating the therapeutic current waveform includes generating a series of voltage spikes.

38. The method of claim 37, wherein generating the series of voltage spikes includes generating a series of alternating polarity constant current voltage spikes having a peak current between 100 and 300 microamps.

39. The method of claim 38, wherein generating the therapeutic current waveform includes generating the series of alternating polarity constant current voltage spikes, each having a peak current of about 200 microamps.

40. The method of claim 36, wherein generating the first therapeutic waveform includes generating a waveform including alternating polarity voltage spikes having a duty cycle below 5%.

41-46. (canceled)

47. The method of claim 24, wherein the first and second electrodes are operatively coupled to vagus nerves on the same lateral side of the human body to cause the therapeutic current to pass along substantially the first vagus nerve.

48. (canceled)

49. The method of claim 24, further comprising:
generating a second therapeutic current waveform to cause current flow between third and fourth electrodes transdermally coupled to first or first and second vagus nerves on laterally opposite sides of the human body respectively opposite to the first and second electrodes.

50-52. (canceled)

53. The method of claim 24, wherein generating the therapeutic current waveform includes generating a plurality of therapeutic current waveforms having different frequencies; and
applying the plurality therapeutic current waveforms as a battery of waveforms in a sequence.

54. (canceled)

55. The method of claim 24, further comprising:
receiving a sensor value into the control circuit from a sensor configured to read an observable characteristic of the human; and
with the control circuit, modifying a current or frequency of the therapeutic current waveform responsive to the sensor value.

56. The method of claim 24, further comprising:
receiving a sensor value into the control circuit from a sensor configured to read an observable characteristic of the human; and
with the control circuit, modifying duration of application of the therapeutic current waveform responsive to the sensor value.

57. The method of claim 24, further comprising:
receiving a user input via an interface into a logic circuit, the user input corresponding to at least one of the group consisting of a condition to be treated and a therapeutic waveform schedule.

58. The method according to claim 57, wherein receiving the therapeutic waveform schedule into the logic circuit includes receiving a waveform frequency.

59. The method according to claim 57, wherein receiving the therapeutic waveform schedule into the logic circuit includes receiving two waveform frequencies; and
further comprising:
loading corresponding parameters into the waveform generator to cause the waveform generator to cooperate with four electrodes electrically coupled to the vagus nerve to apply a first push-pull waveform frequency to the first pair of electrodes and, via the electrical couplings, through portions of two respective vagus nerves, and to apply a second push-pull waveform frequency to the second pair of electrodes, wherein the four electrodes are disposed as cross-body pairs including the first pair and a second pair;
wherein each of two frequencies is determined responsive to the condition to be treated or the therapeutic waveform schedule received into the logic circuit from the interface.

60-72. (canceled)
